(19) United States
(12) Patent Application Publication
(10) Pub. No.: US 2014/0127713 A1
(43) Pub. Date: May 8, 2014

(54) MEANS FOR THE EXAMINATION OF BODY FLUIDS

(71) Applicants: Femke Karina De Theije, Berghem (NL); Albert Hendrik Jan Immink, Eindhoven (NL); Lian Van Lippen, Bladel (NL); Franka Maria Diny Couwenberg, Leenda (NL)

(72) Inventors: Femke Karina De Theije, Berghem (NL); Albert Hendrik Jan Immink, Eindhoven (NL); Lian Van Lippen, Bladel (NL); Franka Maria Diny Couwenberg, Leenda (NL)

(21) Appl. No.: 14/125,100

(22) PCT Filed: Jun. 4, 2012
(86) PCT No.: PCT/IB2012/052797

§ 371 Date: Dec. 10, 2013

(30) Foreign Application Priority Data
Jun. 28, 2011 (EP) 11171728.6

(51) Int. Cl. G01N 33/68

(52) U.S. Cl. CPC G01N 33/6803
USPC 435/7.4; 435/287.2; 436/501; 422/69

(57) ABSTRACT

The invention provides means for making examinations on a sample of body fluid, particularly a sample of blood. The presence of at least one characteristic protein is additionally detected, and the intended examinations are only executed and/or evaluated if said characteristic protein is present. The characteristic protein may for example be human serum albumin or hemoglobin if the body fluid is blood. Preferably, the characteristic protein is quantitatively detected in a competitive assay performed in a cartridge (110) with the help of a sensor device (150).

### FIELD OF THE INVENTION

The invention relates to a method, an apparatus, and a cartridge for making examinations on a sample of body fluid, particularly a sample of blood.

### BACKGROUND OF THE INVENTION

The WO 2008/155716 discloses an optical biosensor in which frustrated total internal reflection (FTIR) of a light beam is detected and evaluated with respect to the amount of target components at the reflection surface. The target components comprise magnetic particles as labels, which allows to affect the processes in the sample by magnetic forces.

### SUMMARY OF THE INVENTION

It is an object of the invention to provide means for increasing the safety of biological examinations made on body fluids like blood.

This object is achieved by a method according to claim 1, an apparatus according to claim 11, and a cartridge according to claim 12. Preferred embodiments are disclosed in the dependent claims.

A method according to the invention relates to the performance of examinations on a sample of body fluid. As an example, the examinations may comprise the point-of-care testing of cardiac markers (e.g. troponin, D-Dimer, procalcitonin, NT proBNP) in human blood, PTH (parathyroid hormone) in human blood, oncology markers in human blood, and melatonin in human blood. The method comprises the following two steps:

a) Detecting if at least one characteristic protein is present in the sample, wherein the term characteristic protein shall by definition indicate that said protein is characteristic of the body fluid to be examined (e.g. blood), either in presence or in concentration in contrast to other biological samples of interest (e.g. urine, saliva). The characteristic protein may hence be a protein that only occurs in the respective body fluid but in no other biological samples, or a protein that occurs in a characteristic amount (concentration) only in the body fluid. Moreover, the combined occurrence of several proteins (perhaps in particular amounts) may be characteristic of the body fluid, qualifying these proteins as characteristic proteins in the sense of the present invention.

b) Carrying out and/or evaluating and/or using the examinations on the sample of body fluid only if said characteristic protein has been detected in the sample.

With other words, the method comprises that an additional test for at least one characteristic protein is made before, during, or after the intended examinations, and that the performance or evaluation of these examinations depends on a positive result of this test.

The invention further relates to an apparatus for making examinations on a sample of body fluid, said apparatus comprising the following components:

a) Means for detecting (additionally to the intended examinations) if a characteristic protein that is characteristic of the body fluid is present in the sample.

b) A control unit that is adapted to prohibit or terminate the performance and/or evaluation and/or use of the intended examinations if no characteristic protein has been detected. The control unit may typically be realized by dedicated electronic hardware, digital data processing hardware with associated software, or a mixture of both.

Furthermore, the invention relates to a cartridge for use in a method or in an apparatus of the kind described above, said cartridge comprising:

a) A sample chamber in which examinations on a sample of body fluid can be carried out.

b) Means that allow to (additionally) detect at least one characteristic protein in a sample provided in the sample chamber.

The cartridge may particularly be a disposable and/or exchangeable container with which a sample of body fluid and/or reagents are provided to the apparatus.

As the method, the apparatus, and the cartridge are related embodiments of the invention, explanations and definitions provided for one of them are analogously valid for the other embodiments, too.

By the provision of an additional test for the presence of a characteristic protein, the invention allows to verify if a given sample that shall be examined is actually a sample of the body fluid of interest. This makes the intended examinations safe against the erroneous processing of a wrong sample. This is particularly important if the examinations shall be made outside a laboratory and/or by nonprofessional personnel, where a mix-up of samples cannot be excluded.

In the following, various preferred embodiments of the invention will be described that relate to both a method, an apparatus, and a cartridge of the kind described above.

As already mentioned, the sample of body fluid may particularly be blood. The characteristic protein will in this case be called “blood protein” in the following.

According to a preferred embodiment of the invention, the characteristic protein (or the characteristic proteins, if several are detected) may be selected from the group comprising hemoglobin, albumins, globulins, immunoglobulins, fibrinogens, alpha 1 antitrypsin, regulatory proteins, lipoproteins, transferrin, C-reactive protein, prothrombin, and MBL. These are examples of “blood proteins” that are characteristic of blood.

In another embodiment of the invention, not only the presence of the characteristic protein is qualitatively detected, but the amount of this protein is determined. This provides additional information that can for example constitute one result among the residual examinations to be performed with the sample.

The aforementioned quantitative determination of the characteristic protein may further be used to increase the reliability of the characteristic protein detection. This means that a positive detection of characteristic protein is only assumed if a given minimal amount of characteristic protein has been determined. Samples other than the body fluid of interest that may comprise small amounts of a characteristic protein can thus be distinguished from actual samples of the body fluid in which the amount of the characteristic protein is above the given threshold.

The characteristic protein may preferably be detected in a competitive assay. This has the advantage that high concentrations of characteristic protein, which are for example characteristic for typical blood proteins like HSA or hemoglobin, can be dealt with without a need to dilute the sample (which might negatively affect the intended examinations). In a competitive assay, the characteristic protein of interest competes with probes for a reagent (e.g. an antibody), wherein the final amount of probes that were able to react with the reagent is evaluated (cf. U.S. 2007/0243563, U.S. 2009/0123376). The higher the amount of characteristic protein, the smaller the resulting signal of a competitive assay will be. The probes may preferably be an analogue or derivative of the characteristic protein.

In a preferred realization of the aforementioned embodiment, the competitive assay comprises probes that are immobilized on a detection surface and antibodies that can bind to the characteristic protein and to the probe (but not to both simultaneously). The amount of binding between probes and antibodies can, at the end of the assay, be determined at the detection surface. This amount is (inversely) related to the amount of characteristic protein in the examined sample.

The aforementioned probes may particularly be immobilized in a detection region on the surface of the sample chamber of the cartridge. Such an embodiment fits to a design of the cartridge in which a plurality of regions or spots on the surface of the sample chamber are coated with different reagents (e.g. antibodies) that provide for different tests. The presence of the characteristic protein can then be detected with the means that are already available for the other examinations.

The antibodies of the described competitive assay may preferably be coupled to (labeled by) magnetic particles. This allows to magnetically actuate these labeled antibodies and their compounds. For instance, compounds between the labeled antibodies and a (blood) protein can be washed away from the detection surface at the end of the competitive assay such that they will not affect measurements at the detection surface. Moreover, the magnetic particles may serve as labels that can readily be detected, for instance to determine how many labeled antibodies have bound to probes.

In general, the sample may be examined by an optical, magnetic, mechanical, acoustic, thermal or electrical procedure, and the cartridge and/or the apparatus may comprise a corresponding sensor unit. A magnetic sensor unit may particularly comprise a coil, Hall sensor, planar Hall sensor, flux gate sensor, SQUID (Superconducting Quantum Interference Device), magnetic resonance sensor, magneto-restrictive sensor, or magneto-resistive sensor of the kind described in the WO 2005/010543 A1 or WO 2005/010542 A2, especially a GMR (Giant Magneto Resistance), a TMR (Tunnel Magneto Resistance), or an AMR (Anisotropic Magneto Resistance). An optical sensor unit may particularly be adapted to detect variations in an output light beam that arise from a frustrated total internal reflection due to target particles at a sensing surface.

### BRIEF DESCRIPTION OF THE DRAWINGS

These and other aspects of the invention will be apparent from and elucidated with reference to the embodiments described hereinafter.

In the drawings:

FIG. 1 schematically shows a side view of an apparatus according to the present invention;

FIG. 2 illustrates a competitive assay as applied in a method according to the invention;

FIG. 3 shows two measured albumin standard curves obtained in a competitive assay for different probe concentrations.

### DETAILED DESCRIPTION OF EMBODIMENTS

Many Point-Of-Care tests are aimed for testing blood, for example tests according to the Magnotech® technology developed by the applicant.

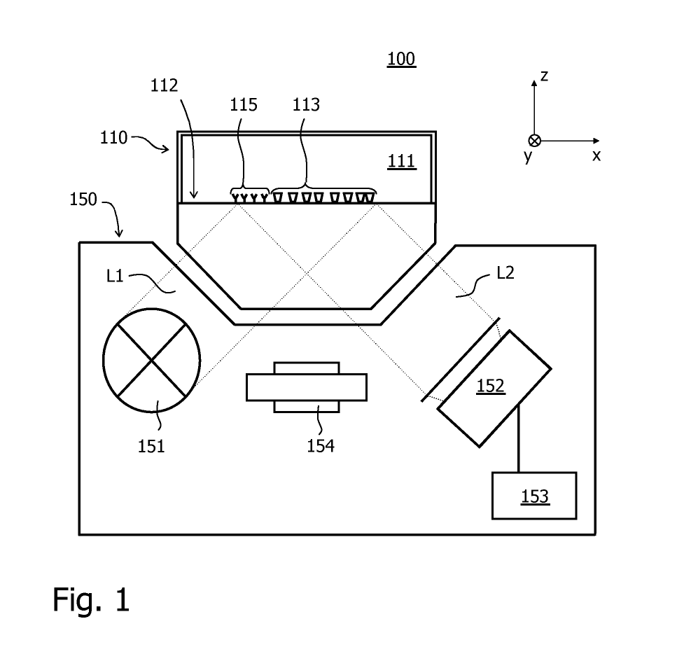

FIG. 1 shows a schematic side view of such a biosensor apparatus 100 that is used for making examinations on blood. The sensor apparatus 100 comprises a reader 150 and a disposable cartridge 110. The cartridge 110 may for example be made from glass or transparent plastic like poly-styrene. It comprises a sample chamber 111 in which a sample of blood with target components to be detected (e.g. cardiac troponin, drugs, antibodies, DNA, parathyroid hormone PTH etc.) can be provided. The sample may further comprise magnetic particles, for example superparamagnetic beads, wherein these particles are usually bound as labels to the aforementioned target components.

The cartridge 110 is transparent with a detection surface 112 that (partially) borders the sample chamber 111. A plurality of detection spots 113 is disposed on the detection surface 112. They comprise binding sites, e.g. antibodies, which can specifically bind the target components.

The reader 150 comprises a light source 151 for emitting an “input light beam” L1, a light detector 152 for detecting and measuring an “output light beam” L2, and an evaluation unit 153 for evaluating the signals of the light detector. The input light beam L1 generated by the light source 151 arrives at the detection surface 112 at an angle larger than the critical angle of total internal reflection (TIR) and is therefore totally internally reflected as the output light beam L2. The output light beam L2 leaves the cartridge 110 and is detected by the light detector, e. g. by the light-sensitive pixels of a camera 153. The light detector 153 thus generates an image of the detection surface, which is further processed in the evaluation unit 153.

The reader 150 further comprises a magnetic field generator, for example electromagnets 154 with a coil and a core disposed at the bottom and/or at the top (not shown) of the cartridge, for controllably generating a magnetic field at the detection surface 112 and in the adjacent space of the sample chamber 111. With the help of this magnetic field, the magnetic particles can be manipulated, i.e. be magnetized and particularly be moved (if magnetic fields with gradients are used). Thus it is for example possible to attract magnetic particles to the detection surface 112 in order to accelerate the binding of the associated target component to said surface.

The described sensor apparatus 100 applies optical means for the detection of magnetic particles and the target components one is actually interested in. For eliminating or at least minimizing the influence of background, the detection technique should be surface-specific. As indicated above, this is achieved by using the principle of frustrated total internal reflection (FTIR). This principle is based on the fact that an evanescent wave propagates (exponentially dropping) into the sample chamber 111 when the incident light beam L1 is totally internally reflected. If this evanescent wave then interacts with another medium having a different refractive index from water like the magnetic particles, part of the input light will be coupled into the sample fluid (this is called “frustrated total internal reflection”), and the reflected intensity will be reduced (while the reflected intensity will be 100% for a clean interface and no interaction). Further details of this procedure may be found in the WO 2008/115723 A1, which is incorporated into the present text by reference.

If sample media other than blood are erroneously used in the apparatus 100, the intended blood examinations will give an incorrect result. It is therefore desirable to have a check on the use of the correct sample.

To address this issue, a biological test on characteristic proteins that are unique for blood is proposed, for example on hemoglobin and/or human serum albumin (HSA). Other fluids than body fluids will not contain such characteristic proteins. For other body fluids than blood, characteristic proteins can be present (e.g. for diabetes patients in urine, or periodontology patients in saliva), however the concentrations in blood will be higher than in other body fluids (for blood the normal HSA concentration is 3.5 to 5 g/dL). By adding a quantitative assay test in a cartridge or biosensor apparatus that covers the characteristic proteins, a unique matrix indicator can be provided.

Important groups of characteristic blood proteins that can be tested on comprise:
albumins (which create oncotic pressure and transport other molecules; typical level in blood: 3.5-5 g/dL),
immunoglobins (which participate in immune system; typical level in blood: 1.0-1.5 g/dL);
fibrinogens (which participate in blood coagulation; typical level in blood: 0.2-0.45 g/dL);
alpha 1 antitrypsin (which neutralizes trypsin that has leaked from the digestive system);
regulatory proteins (which regulate gene expression);
transferrin.

In the biosensor apparatus 100 of FIG. 1, the proposed characteristic protein assay is realized by an additional binding spot 115 on the detection surface 112 of the cartridge 110 together with corresponding modifications of the reader 150. In particular, the evaluation unit 153 of the reader 150 is adapted to evaluate the measurement signals of the light detector 152 with respect to the presence of a characteristic protein in the examined sample and to control the residual examinations and/or their evaluation according to the results of this evaluation. Namely, if a desired characteristic protein is not detected (not at all or not in a specified minimal amount), this is taken as an indication that the sample is no blood and that it should therefore be rejected. A corresponding warning may be emitted to the user.

The applied characteristic protein assay may preferably be in a competitive format since the concentration of the characteristic proteins is usually too high to be measured in e.g. a sandwich assay, and since sample pre-dilution is not allowed.

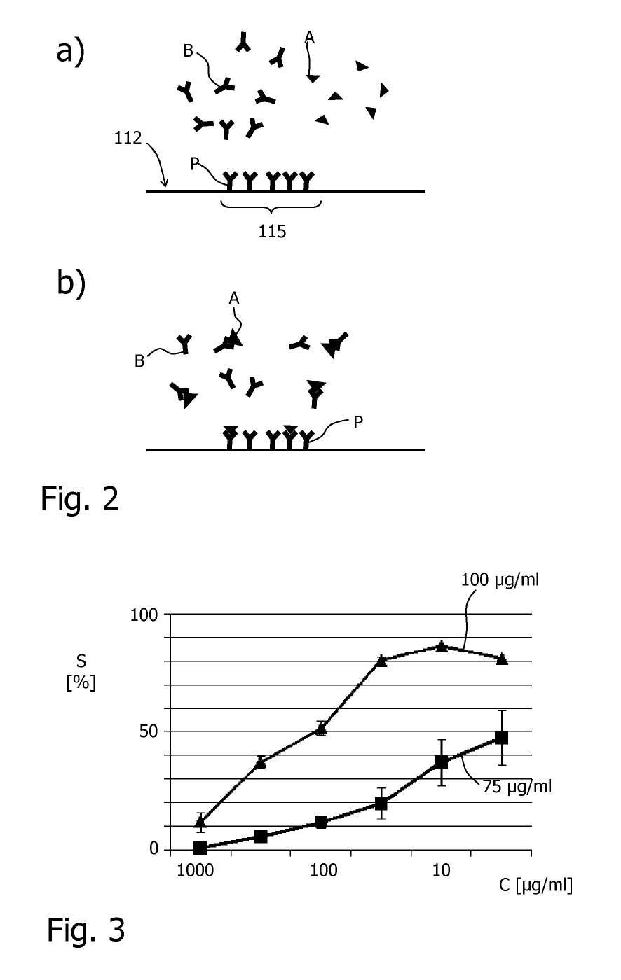

FIG. 2 schematically illustrates the principle of a competitive assay. The additional binding spot 115 on the detection surface 112 of the cartridge is coated in this case with a probe protein P, e.g. HSA or an analogue. The characteristic protein B of interest (e.g. HSA) is in the blood sample above this binding spot. When a suitable reagent like an antibody A is added (FIG. 2a), the probes P compete with the characteristic protein B in the sample for the binding of this reagent A. After the reactions have taken place, the reagent will be bound to the probes P in an amount that inversely depends on the amount of characteristic protein B in the sample (FIG. 2b).

As an example of a competitive assay in a point-of care-setting, albumin antibodies were coupled to 500 nm Ademtech COOH-terminated superparamagnetic beads, and they were dried onto a region of a cartridge (providing a reservoir of reagent A). Human albumin was then printed as a probe P onto the detection surface 112 in an additional binding spot 115, whereby the print concentration determines the sensitivity of the assay. This is illustrated in FIG. 3, in which the measured sensor signal S (relative units) is shown in dependence on the concentration c of the characteristic protein B (albumin, horizontal axis), wherein the two curves correspond to print concentrations of 75 μg/ml and 100 μg/ml, respectively.

The parts of the cartridge were then taped together to close, and assay buffer with known concentrations of human albumin (HSA) was entered into the sample chamber. The standard assay range in buffer, about 10-1000 μg/ml, could be met with an actuation protocol (assay time) of 5 minutes.

In summary, the present invention provides means that allow to check in a biosensor apparatus if the correct matrix (e.g. blood) is used. A biological test on proteins that are unique for blood, e.g. hemoglobin or human serum albumin, is used for this purpose. By adding a qualitative or quantitative assay test in the cartridge that covers the characteristic proteins, a unique matrix indicator can thus be provided.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

1. A method for making examinations on a sample of body fluid by an apparatus for making examinations on the sample of body fluid, comprising the following steps:
a) detecting by the apparatus and in a competitive assay if at least one blood protein (B) is present in the sample, said blood protein (B) being selected from the group consisting of hemoglobin, albumins, globulins, immunoglobulins, fibrinogens, alpha 1 antitrypsin, regulatory proteins, lipoproteins, transferrin, C-reactive protein, prothrombin, and MBL;
b) carrying out and/or evaluating and/or using the examinations on the sample by The apparatus only if said blood protein has been detected in the sample.

2. (canceled)

3. (canceled)

4. (canceled)

5. The method according to claim 1, characterized in that the amount of characteristic protein is determined.

6. The method according to claim 5, characterized in that a detection of the presence of characteristic protein (B) is only assumed if a given minimal amount of the characteristic protein has been determined.

7. The method according to claim 1, characterized in that the characteristic protein (B) is detected in a competitive assay.

8. The method according to claim 7, characterized in that the competitive assay comprises probes (P) that are immobilized on a detection surface and antibodies (A) that competitively bind to the characteristic protein (B) and said probes (P).

9. The method according to claim 8, characterized in that the antibodies (A) are coupled to magnetic particles.

10. The method according to claim 1, characterized in that the sample is examined by an optical, magnetic, mechanical, acoustic, thermal or electrical procedure.

11. An apparatus for making examinations on a sample of a body fluid comprising:
a) means for additionally detecting if at least one characteristic protein (B) is present in the sample which is characteristic of the body fluid;
b) a control unit that is adapted to prohibit and/or terminate the performance and/or evaluation and/or use of the examinations if no characteristic protein has been detected.

12. (canceled)

13. (canceled)

14. (canceled)

15. (canceled)
